(19) United States
(12) Patent Application Publication
(10) Pub. No.: US 2015/0241355 A1
(43) Pub. Date: Aug. 27, 2015

(54) APPARATUS FOR PERFORMING SPECTROSCOPY HAVING A PARABOLIC REFLECTOR AND SERS ELEMENTS

(71) Applicants: Shih-Yuan Wang, Palo Alto, CA (US); Gary Gibson, Palo Alto, CA (US); Zhiyong Li, Foster City, CA (US); Alexandre M. Bratkovski, Mountain View, CA (US); Steven Barcelo, Palo Alto, CA (US); Ansoon Kim, Mountain View, CA (US); Mineo Yamakawa, Palo Alto, CA (US); Zhang-Lin Zhou, San Diego, CA (US); Huei Pei Kuo, Cupertino, CA (US)

(72) Inventors: Shih-Yuan Wang, Palo Alto, CA (US); Gary Gibson, Palo Alto, CA (US); Zhiyong Li, Foster City, CA (US); Alexandre M. Bratkovski, Mountain View, CA (US); Steven Barcelo, Palo Alto, CA (US); Ansoon Kim, Mountain View, CA (US); Mineo Yamakawa, Palo Alto, CA (US); Zhang-Lin Zhou, San Diego, CA (US); Huei Pei Kuo, Cupertino, CA (US)

(73) Assignee: HEWLETT-PACKARD DEVELOPMENT COMPANY, L.P., Houston, TX (US)

(21) Appl. No.: 14/406,653

(22) PCT Filed: Jul. 31, 2012
(86) PCT No.: PCT/US2012/049031

§ 371 Date: Dec. 9, 2014

(51) Int. Cl. G01N 21/65; A61F 2/02; A61F 2/82; G02B 5/10

(52) U.S. Cl. CPC G01N 21/658; G02B 5/10; A61F 2/02; A61F 2/82; G01N 2201/068

(57) ABSTRACT

According to an example, an apparatus for performing spectroscopy includes a parabolic reflector and a plurality of surface-enhanced Raman spectroscopy (SERS) elements spaced from the parabolic reflector and positioned substantially at a focal point of the parabolic reflector. The parabolic reflector is to reflect Raman scattered light emitted from molecules in a near field generated by the plurality of SERS elements to substantially increase the flux of the Raman scattered light emitted out of the apparatus.

### BACKGROUND

In surface-enhanced Raman scattering (SERS), vibrationally excitable levels of an analyte are probed. The energy of a photon shifts by an amount equal to that of the vibrational level excited by the photon (Raman scattering). A Raman spectrum, which consists of a wavelength distribution of bands corresponding to molecular vibrations specific to the analyte being probed, may be detected to identify the analyte. In SERS, the analyte molecules are in close proximity, for instance, less than tens of nanometers, to metal nano-particles that may be or may not be coated with a dielectric, such as silicon dioxide, silicon nitride, and a polymer, that, once excited by light, set up plasmon modes, which create near fields around the metal nano-particles. These fields can couple to analyte molecules in the near field regions. As a result, concentration of the incident light occurs at close vicinity to the nano-particles, enhancing the Raman scattering from the analyte molecules.

SERS has recently been performed to probe fluids in a specimen in vivo through implantation of the metal nano-particles subcutaneously. However, because light to excite the metal nanoparticles is reflected directly from the specimen's surface, such as the specimen's skin, and the Raman scattered light is degraded by the Raman light generated from the specimen's surface, accurate results were difficult to obtain. One technique to overcome some of these issues is to detect the enhanced Raman signal as a function of spatially offset distance from the pump beam. This “offset” technique enables the deconvolution of the Raman signal from the scattered excitation light from the surface (e.g., skin) but is time-consuming and complicated to implement.

### BRIEF DESCRIPTION OF THE DRAWINGS

Features of the present disclosure are illustrated by way of example and not limited in the following figure(s), in which like numerals indicate like elements, in which:

FIG. 1 shows a perspective view of an apparatus for performing spectroscopy, according to an example of the present disclosure;

FIG. 2 shows a side view of the apparatus for performing spectroscopy depicted in FIG. 1, according to an example of the present disclosure;

FIGS. 3A and 3B, respectively show cross-sectional side views of the apparatus depicted in FIG. 2, according to examples of the present disclosure;

FIG. 4A shows an isometric view of an array of SERS elements, in this instance nano-fingers, that may be implemented in the apparatus depicted in FIGS. 1-3B, according to an example of the present disclosure;

FIGS. 4B and 4C, respectively show cross-sectional views along a line A-A, shown in FIG. 4A, prior to and following collapse of the nano-fingers, according to examples of the present disclosure; and

FIG. 5 shows a flow diagram of a method for fabricating an apparatus for performing spectroscopy, according to an example of the present disclosure.

### DETAILED DESCRIPTION

For simplicity and illustrative purposes, the present disclosure is described by referring mainly to an example thereof. In the following description, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure.

Throughout the present disclosure, the terms “a” and “an” are intended to denote at least one of a particular element. As used herein, the term “includes” means includes but not limited to, the term “including” means including but not limited to. The term “based on” means based at least in part on. In addition, the term “light” refers to electromagnetic radiation with wavelengths in the visible and non-visible portions of the electromagnetic spectrum, including infrared, near infrared, and ultra-violet portions of the electromagnetic spectrum.

Disclosed herein are an apparatus for performing spectroscopy and a method for fabricating the apparatus. The apparatus disclosed herein comprises a parabolic reflector and a plurality of surface-enhanced Raman spectroscopy (SERS) elements spaced from the parabolic reflector and positioned substantially at a focal point of the parabolic reflector. The parabolic reflector is to substantially increase the flux of the Raman signal or scattered light emitted from molecules around the SERS elements that is directed to the detector. In addition, the parabolic reflector is to be tilted with respect to the direction in which an excitation light is applied onto the SERS elements to substantially prevent specularly reflected or low-angle scattered excitation light from reaching the detector and competing with the Raman signal.

The apparatus disclosed herein may include an off-axis parabolic reflector, in which the SERS elements are positioned substantially at a focal point of the off-axis parabolic reflector. In one regard, the off-axis parabolic reflector enables the excitation light to be introduced onto the SERS elements from a direction that minimizes collection of the excitation light by the detector so that the excitation light does not substantially interfere with the Raman signals emitted from the molecules in the near fields of the SERS elements. In addition, or alternatively, the apparatus disclosed herein may include a back reflector that is positioned to reflect the Raman signals emitted from the molecules in the near fields of the SERS elements toward the parabolic reflector, to thereby further increase the flux of the Raman signals directed to the detector. According to an example, the back reflector is incorporated into the SERS element. In addition, in instances where the excitation region is small compared to the focal length of the parabolic reflector, then the back reflector may be substantially smaller than the parabolic reflector and may be flat. For instance, the back reflector may be incorporated into the substrate of a flat SERS element.

The apparatus disclosed herein may be fabricated or integrated with a housing that is to be implanted into a specimen, such as a human, an animal, an insect, a plant, a non-living item, etc. According to an example, the housing forms part of an implantable device, such as a surgically implantable stent. In one regard, the apparatus disclosed herein may be implemented to generate relatively high intensity/flux (or signal strength) and accurately detectable Raman signals in in-vivo Raman spectroscopy operations. In other words, the apparatus disclosed herein enables Raman signals having higher intensities/fluxes to be detected as compared with conventional in vivo Raman spectroscopy techniques that employ conventional spatially offset techniques to detect the Raman signals.

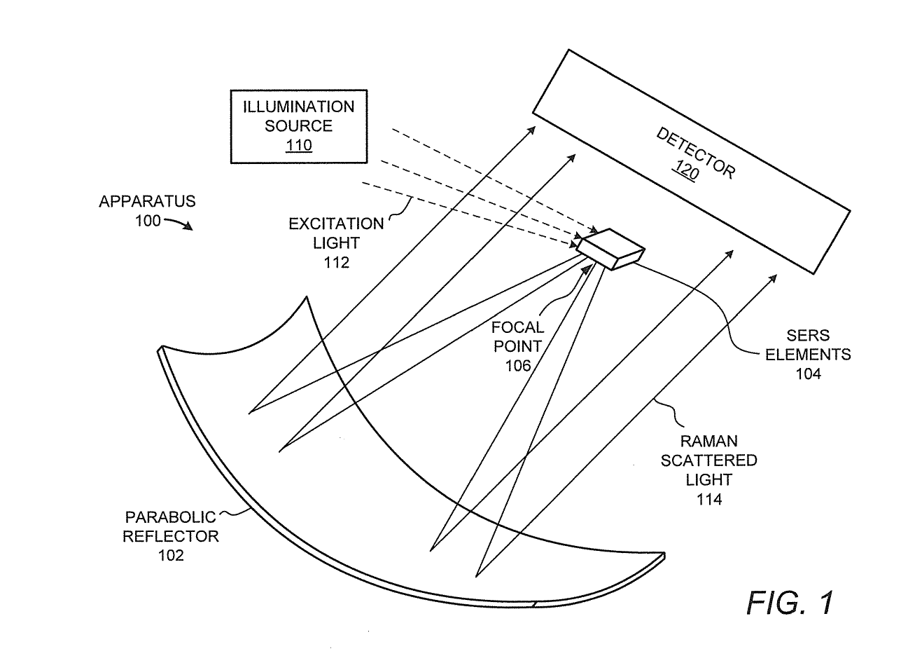

With reference first to FIG. 1, there is shown a perspective view of an apparatus 100 for performing spectroscopy, according to an example. It should be understood that the apparatus 100 depicted in FIG. 1 may include additional components and that some of the components described herein may be removed and/or modified without departing from a scope of the apparatus 100. It should also be understood that the components depicted in FIG. 1 are not drawn to scale and thus, the components may have different relative sizes with respect to each other than as shown therein.

The apparatus 100 may be implemented to perform spectroscopy, which is also equivalently referred herein as surface-enhanced Raman spectroscopy (SERS), to detect a molecule in an analyte sample with a relatively high level of sensitivity. As a particular example, the apparatus 100 is to be implemented to perform spectroscopy following implantation of the apparatus 100 into a specimen, such as a human, an animal, an insect, a plant, non-living item, etc., or in any gaseous or liquid or liquid crystal environment where surface scattering degrades the signal. Examples of such environments include, for instance, chemical/food processing facilities, where surfaces and any interstitial contribution to the signal is not desirable and need to be minimized. The apparatus 100 may thus be implemented to analyze molecules in a fluid in a specimen, such as blood, lymph, saliva, interstitial fluid, an analyte carried by gas, air flow, etc. The apparatus 100 may alternatively be implemented in spectroscopy applications that do not involve implantation of the apparatus 100.

As shown, the apparatus 100 includes a parabolic reflector 102 and SERS elements 104 (a single substrate on which the SERS elements 104 are positioned is depicted in FIG. 1). The parabolic reflector 102 generally comprises a reflective device having a paraboloidal shaped reflective surface, in which a spherical wave generated by a point source in the focal point 106 of the parabolic reflector 102 is transformed into a plane wave propagating as a collimated beam along an axis. The SERS elements 104, which comprise Raman-enhancing elements, such as plasmonic nano-particles or other Raman-enhancing structures, arranged in various ordered or random configurations on a substrate, are depicted as being positioned substantially at or near the focal point 106 of the parabolic reflector 102. The SERS elements 104 may one or both of enhance Raman scattering and facilitate analyte adsorption. The SERS elements 104 generally enhance sensing operations, such as, surface enhanced Raman spectroscopy (SERS), enhanced photoluminescence, etc., to be performed on molecules positioned on or near the SERS elements 104.

In some examples, the SERS elements 104 may be functionalized to facilitate adsorption of analyte molecules. For example, surfaces of the SERS elements 104 may be functionalized such that a particular class of analytes is attracted and may bond or be preferentially adsorbed onto the SERS elements 104.

According to an example, when an illumination source 110 emits an excitation light 112 (or equivalently, a pump light), such as a laser beam, an LED beam, etc., onto the SERS elements 104, the SERS elements 104 create near fields around the SERS elements 104. Although the excitation light 112 has been depicted as being directly emitted from the illumination source 110 onto the SERS elements 104, it should be understood that various optical components, e.g., mirrors, prisms, lenses etc., may be positioned to direct the excitation light 112 to the SERS elements. In any regard, the near fields around the SERS elements 104 couple to analyte molecules (not shown) in the vicinities of the SERS elements 104. The metallic nanoparticles (or other plasmonic structures) of the SERS elements 104 also act to enhance the Raman emission process of the analyte molecules. As a result, Raman scattered light is emitted from the analyte molecules and the emission of the Raman scattered light is enhanced by the SERS elements 104. A portion of the Raman scattered light 114, which may be emitted in all directions from the analyte molecules near the SERS elements 104, is emitted toward the parabolic reflector 102. The Raman scattered light 114 is reflected from a reflective surface of the parabolic reflector 102 into a collimated or nearly collimated beam, as depicted in FIG. 1. For instance, the parabolic reflector 102 may focus the Raman scattered light 114 onto an optical device, such as an aperture, a lens, etc., to direct the Raman scattered light 114 toward a detector 120.

As also depicted in FIG. 1, the detector 120 is positioned to collect the Raman scattered light 114 reflected from the reflective surface of the parabolic reflector 102 (and in various instances, Raman scattered light 114 that is directly emitted from the molecules). Alternatively, various optical components, e.g., mirrors, prisms, etc., may be positioned to direct the Raman scattered light 114 to the detector 120, which may comprise a spectrometer, photodetectors, etc. In any regard, the detector 120 is to generate electrical signals corresponding to the wavelengths of light contained in the detected Raman scattered light 114, which may be processed to determine the Raman spectrum of the Raman scattered light 114 originating from the analyte molecules near the SERS elements 104.

According to an example, the excitation light 112 is to be emitted through a surface layer (not shown), for instance, a skin layer, body tissue, vein walls, cover, etc., under which the apparatus 100 is implanted. In another example, the excitation light 112 is to be emitted through a gaseous or liquid environment in which the apparatus 100 has been positioned. In addition, the Raman scattered light 114 is to be emitted through the surface layer and/or the gaseous or liquid environment. According to an example, the location on the surface layer and/or the gaseous or liquid environment at which the Raman scattered light 114 is emitted is offset with respect to the location on the surface layer and/or in the gaseous or liquid environment at which the excitation light 112 is emitted. In one regard, therefore, contribution of the excitation light 112 that is scattered from the surface layer and/or the gaseous or liquid environment in the light detected by the detector 120 may substantially be minimized. In other words, the parabolic reflector 102 is tilted with respect to the direction in which the excitation light 112 is applied onto the SERS elements 104, for instance, to minimize the surface contributions of the excitation light 112 scattered (which may include spurious Raman signals from the surface and/or the interstitial gaseous or liquid environment) into the Raman scattered light 114 directed into the detector 120.

The apparatus 100 has a size, a configuration, and is fabricated of materials that make the apparatus 100 suitable for implantation into a specimen. According to an example, and as discussed in greater detail below, the apparatus 100 comprises a surgically implantable stent. The parabolic reflector 102 may comprise a polymer material that has been nanoimprinted, molded, and/or stamped to form the shape of the parabolic reflector 102. In addition, a surface of the polymer material is coated with at least one of a metal, such as gold, silver, etc., a protective coating of dielectric material, Bragg layers, etc., to be reflective. Moreover, the parabolic reflector 102 may have dimensions that range from about less than a millimeter to about a couple of centimeters. In addition, the SERS elements 104 may be positioned on a substrate that is in the range of about 100 microns wide or larger.

The SERS elements 104 comprise Raman-enhancing elements, such as plasmonic nanoparticles or nanostructures, which may comprise plasmon-supporting materials such as but not limited to, gold (Au), silver (Ag), and copper (Cu). The Raman-enhancing elements may have nanoscale surface roughness, which is generally characterized by nanoscale surface features on the surface of the layer(s) and may be produced spontaneously during deposition of the plasmon-supporting material layer(s). By definition herein, a plasmon-supporting material is a material that facilitates Raman scattering and the production or emission of the Raman signal from an analyte on or near the material during Raman spectroscopy. In addition, the Raman-enhancing elements of the SERS elements 104, e.g., plasmonic nanostructures, may be deposited onto a substrate formed, for instance, of a polymer material, a metallic material, a semiconductor material, etc., through, for instance, physical vapor deposition (PVD), chemical vapor deposition (CVD), sputtering, etc., of metallic material, or self-assembly of pre-synthesized nano-particles.

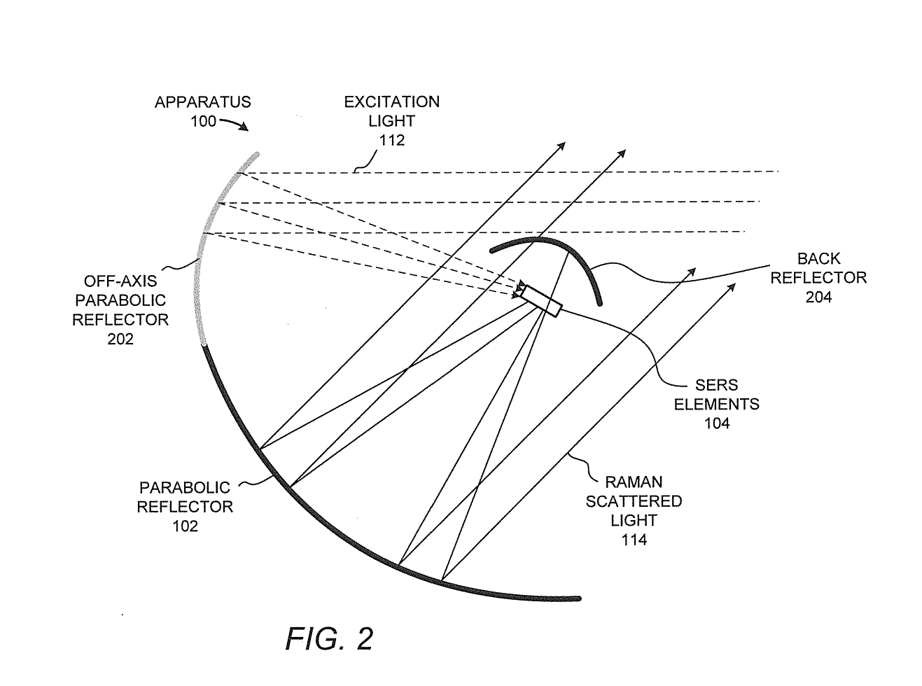

Turning now to FIG. 2, there is shown a side view of the apparatus 100 according to another example. It should be understood that the apparatus 100 depicted in FIG. 2 may include additional components and that some of the components described herein may be removed and/or modified without departing from a scope of the apparatus 100. It should also be understood that the components depicted in FIG. 1 are not drawn to scale and thus, the components may have different relative sizes with respect to each other than as shown therein.

As shown in FIG. 2, the apparatus 100 includes the parabolic reflector 102 and the SERS elements 104. In addition, the apparatus 100 is depicted as including an off-axis parabolic reflector 202 and a back reflector 204, which may comprise a spherical reflector, a parabolic reflector, a flat reflector, etc. It should be understood that the back reflector 204 may be removed from the apparatus 100 without departing from a scope of the apparatus 100 disclosed herein.

The off-axis parabolic reflector 202 is to focus the excitation light 112 onto the SERS elements 104 as shown in FIG. 2. Thus, the off-axis parabolic reflector 202 is positioned and configured such that the SERS elements 104 are located at or near the focal point of the off-axis parabolic reflector 202. In addition, the off-axis parabolic reflector 202 is positioned and configured to minimize the spurious signal generated at the surface and/or by the interstitial gaseous or liquid environment by the excitation light 112 being scattered therefrom from being directed toward a detector 120 (not shown).

As shown in FIG. 2, the off-axis parabolic reflector 202 is attached to and integrally formed with the parabolic reflector 102. In this regard, the off-axis parabolic reflector 202 may be formed into the same polymer block as the parabolic reflector 102. Alternatively, the off-axis parabolic reflector 202 may be formed separately from the parabolic reflector 102. In this example, the off-axis parabolic reflector 202 may be separate from and spaced from the parabolic reflector 202. In any regard, the off-axis parabolic reflector 202 may be formed of similar materials, similar dimensions, and through similar processes as the parabolic reflector 102 discussed above.

The back reflector 204 is positioned and configured to reflect Raman scattered light 114 emitted from molecules near the SERS elements 104 to be directed to the parabolic reflector 102. In this regard, the back reflector 204 generally functions to increase the intensity or flux of the Raman scattered light 114 directed to the detector 120 by directing more light toward the parabolic reflector 102. The back reflector 204 may also be formed of similar materials, similar dimensions, and through similar processes as the parabolic reflector 102 discussed above.

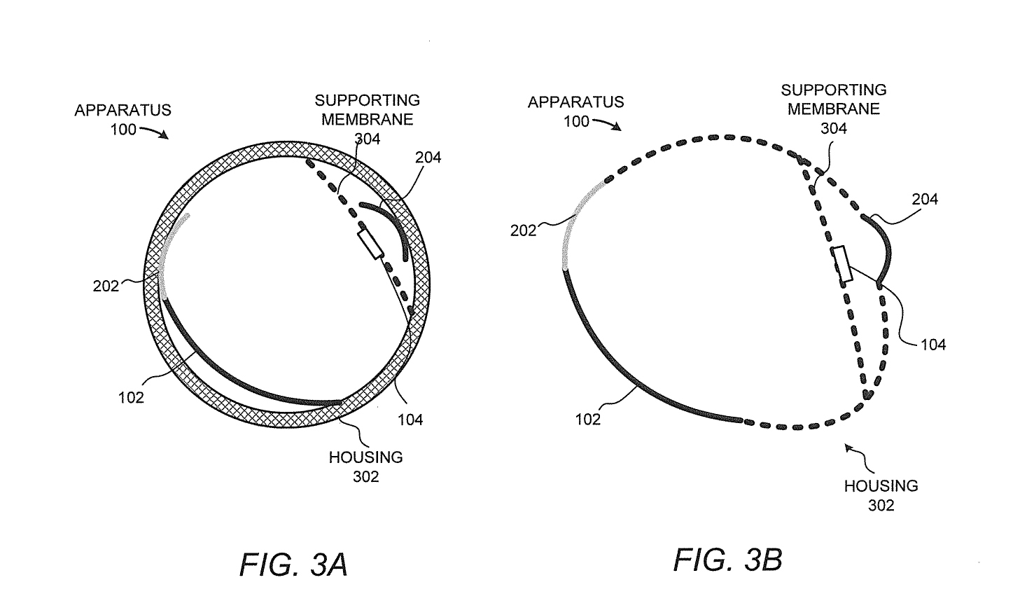

Turning now to FIGS. 3A and 3B, there are respectively shown cross-sectional side views of the apparatus 100, in which the apparatus 100 is formed as an integrated component, according to two examples. It should be understood that the apparatus 100 depicted in FIGS. 3A and 3B may include additional components and that some of the components described herein may be removed and/or modified without departing from a scope of the apparatus 100. For instance, either or both of the off-axis parabolic reflector 202 and the back reflector 204 may be removed. It should also be understood that the components depicted in FIGS. 3A and 3B are not drawn to scale and thus, the components may have different relative sizes with respect to each other than as shown therein.

Generally speaking, the apparatus 100 depicted in FIGS. 3A and 3B may have relatively elongated configurations and sizes, similar to implantable stents. In addition, the ends of the apparatus 100 may be open or have a plurality of openings to enable fluid of a specimen, such as interstitial fluid, blood, lymph, saliva, etc., to flow into the apparatus 100, for instance, through capillary forces. In addition, although the parabolic reflector 102 has been depicted as being arranged transverse to the longitudinal axis of the apparatus 100, the parabolic reflector 102 may instead be arranged parallel to the longitudinal axis.

The apparatus 100 may include a single set of the parabolic reflector 102 and the SERS elements 104. In this example, the remaining sections of the apparatus 100 that do not include the set of the parabolic reflector 102 and the SERS elements 104 may have a cylindrical configuration. The apparatus 100 may alternatively include multiple sets of parabolic reflectors 102 and the SERS elements 104 positioned along the housing 302. In this example, the sets of the parabolic reflectors 102 and the SERS elements 104 may be the same or may differ from each other, for instance, by including an off-axis parabolic reflector 202 in one of the sets while omitting the off-axis parabolic reflector 202 from another one of the sets. In addition, the sections of the apparatus 100 between the sets may have a cylindrical configuration.

With reference first to FIG. 3A, the apparatus 100 is depicted as including a housing 302, within which the parabolic reflector 102, the SERS elements 104, the off-axis parabolic reflector 202, and the back reflector 204 are positioned. The housing 302 may comprise any material suitable for implantation into a specimen, such as silicon, polymer, plastic, silver, titanium, etc. The housing 302 may also comprise other materials, such as materials that may be toxic to a specimen, in instances where the apparatus 100 is to be implemented without being implanted into a specimen. In addition, the housing 302 is formed to enable excitation light 112 and Raman scattered light 114 to be transmitted therethrough, for instance, as shown in FIG. 2. In this regard, the housing 302 is formed with a plurality of holes, for instance, as a mesh structure. In addition, or alternatively, the housing 302 is formed of an optically transparent material.

Additionally, in FIG. 3A, the SERS elements 104 are depicted as being supported in place, i.e., at or near the focal point of the parabolic reflector 102, by a supporting membrane 304. The supporting membrane 304 may also be formed to enable light to be transmitted therethrough and may be formed of the same or similar materials as the housing 302. Alternatively, however, the SERS elements 104 may be attached to the back reflector 204 by the supporting membrane 304 or another structure.

Turning now to FIG. 3B, the apparatus 100 is depicted with the parabolic reflector 102, the off-axis parabolic reflector 202, and the back reflector 204 connected together to form a housing 302. In this regard, the parabolic reflector 102, the off-axis parabolic reflector 202, and the back reflector 204 are integrated into the housing 302. In this example, the portions of the housing 302 between the parabolic reflector 102 and the back reflector 204 may be formed to enable light to be transmitted therethrough and may be formed of the same or similar materials as the housing 302 as discussed above with respect to FIG. 3A. In addition, the supporting membrane 304 that supports the SERS elements 104 may be formed of any of the same materials as the supporting membrane 304 discussed above with respect to FIG. 3A.

As a further alternative, in both FIGS. 3A and 3B, the SERS elements 104 may be integrated with the back reflector 204, such that, for instance, the back reflector 204 comprises the substrate on which the Raman-enhancing elements of the SERS elements 104 are provided.

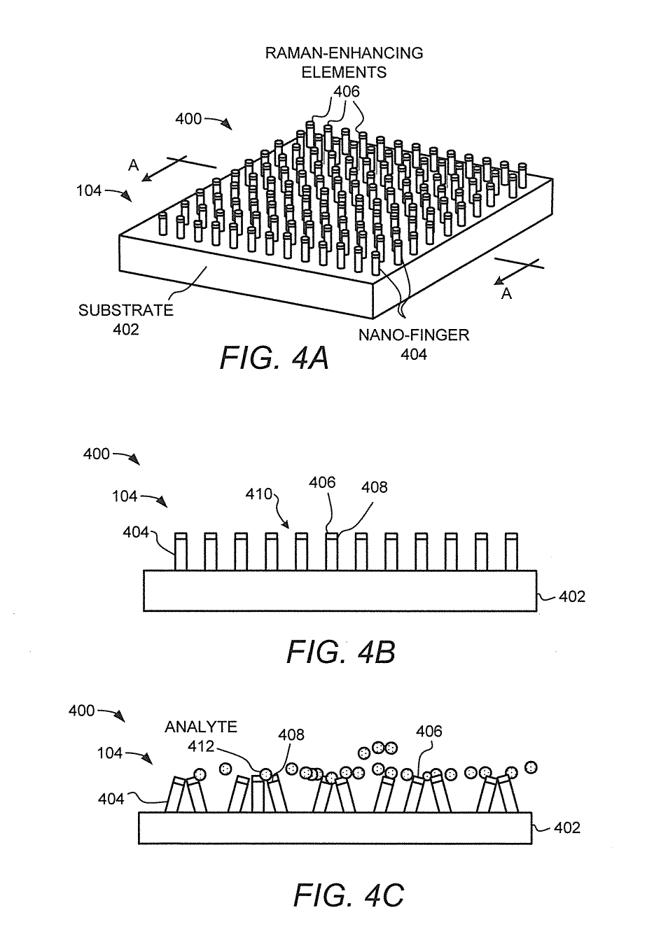

Turning now to FIG. 4A, there is shown an isometric view of an array 400 of SERS elements 104, in this instance Raman-enhancing elements 406 positioned on tops of nano-fingers 404, depicted in FIGS. 1-3B, according to an example. As shown in FIG. 4A, the array 400 includes a substrate 402 upon which the nano-fingers 404 extend. More particularly, the nano-fingers 404 are depicted as being attached to and extending above a surface of the substrate 402. The substrate 402 may be formed of any suitable material, such as, silicon, silicon nitride, glass, plastic, polymer, SiO₂, Al₂O₃, aluminum, etc., or a combination of these materials, etc.

According to an example, the nano-fingers 404 are formed of a relatively flexible material to enable the nano-fingers 404 to be laterally bendable or collapsible, for instance, to enable tips of the nano-fingers 404 to move toward each other, as discussed in greater detail herein below. Examples of suitable materials for the nano-fingers 404 include polymer materials, such as, UV-curable or thermal curable imprinting resist, polyalkylacrylate, polysiloxane, polydimethylsiloxane (PDMS) elastomer, polyimide, polyethylene, polypropelene, polyurethane, fluoropolymer, etc., or any combination thereof, metallic materials, such as, gold, silver, aluminum, etc., semiconductor materials, etc., and combinations thereof.

The nano-fingers 404 are attached to the surface of the substrate 402 through any suitable attachment mechanism. For instance, the nano-fingers 404 are grown directly on the substrate 402 surface through use of various suitable nano-structure growing techniques. As another example, the nano-fingers 404 are integrally formed with the substrate 402. In this example, for instance, a portion of the material from which the substrate 402 is fabricated is etched or otherwise processed to form the nano-fingers 404. In a further example, a separate layer of material is adhered to the substrate 402 surface and the separate layer of material is etched or otherwise processed to form the nano-fingers 404. In various examples, the nano-fingers 404 are fabricated through a nanoimprinting or embossing process in which a template of relatively rigid pillars is employed in a multi-step imprinting process on a polymer matrix to form the nano-fingers 404. In these examples, a template may be formed through photolithography or other advanced lithography with the desired patterning to arrange the nano-fingers 404 in the predetermined arrangement. More particularly, for instance, the desired patterns may be designed on a mold by any of E-beam lithography, photolithography, laser interference lithography, Focused Ion Beam (FIB), self-assembly of spheres, etc. In addition, the pattern may be transferred onto another substrate, for instance, a silicon, glass, or polymer substrate (PDMS, polyimide, polycarbonate, etc.). Various other processes, such as, etching, and various techniques used in the fabrication of micro-electromechanical systems (MEMS) and nano-electromechanical systems (NEMS) may also be used to fabricate the nano-fingers 404.

A nano-finger 404 is defined, for instance, as an elongated, nanoscale structure having a length (or height) that exceeds by more than several times a nanoscale cross sectional dimension (for instance, width) taken in a plane perpendicular to the length (for instance, length>3×width). In general, the length is much greater than the width or cross sectional dimension to facilitate bending of the nano-finger 404 laterally toward one or more neighboring nano-fingers 404. In some examples, the length exceeds the cross sectional dimension (or width) by more than a factor of about 5 or 10. For example, the width may be about 100 nanometers (nm) and the height may be about 500 nm. In another example, the width at the bases of the nano-fingers 404 may range between about 10 nm and about 1 micrometer (μm) and the length may range between about 50 nm and 2 μm. In other examples, the nano-fingers 404 are sized based upon the types of materials used to form the nano-fingers 404. Thus, for instance, the more rigid the material(s) used to form the nano-fingers 404, the less the width of the nano-fingers 404 may be to enable the nano-fingers 404 to be laterally collapsible. In further examples, the nano-fingers 404 may form ridges in which two of three dimensions (for instance, length and height) exceed by more than several times a nanoscale cross sectional dimension (for instance, width).

The nano-fingers 404 have been depicted as having substantially cylindrical cross-sections. It should, however, be understood that the nano-fingers 404 may have other shaped cross-sections, such as, for instance, rectangular, square, triangular, etc. In addition, or alternatively, the nano-fingers 404 may be formed with one or more features, such as, notches, bulges, etc., to substantially cause the nano-fingers 404 to be inclined to collapse in particular directions. Thus, for instance, two or more adjacent nano-fingers 404 may include the one or more features to increase the likelihood that the nano-fingers 404 collapse toward each other. Various manners in which the nano-fingers 404 may be collapsed are described in greater detail herein below.

The array 400 includes a substantially random distribution of nano-fingers 404 or a predetermined configuration of nano-fingers 404. In any regard, according to an example, the nano-fingers 404 are arranged with respect to each other such that the tips of at least two neighboring nano-fingers 404 are able to be brought into close proximity with each other when the nano-fingers 404 are in a collapsed state, for instance, less than about 10 nanometers apart from each other. By way of particular example, the neighboring nano-fingers 404 are positioned less than about 100 nanometers apart from each other. According to a particular example, the nano-fingers 404 are patterned on the substrate 402 such that neighboring ones of the nano-fingers 404 preferentially collapse into predefined geometries, for instance, triangles, squares, pentagons, etc.

In addition, although FIG. 4A depicts the array as having a relatively large number of nano-fingers 404 arranged along each row, it should be understood that the array may include any number of nano-fingers 404 in each row. In one regard, a relatively large number of nano-fingers 404 is provided on the substrate 402 to generally enhance the likelihood of detectable Raman scattering from molecules of an analyte.

The Raman-enhancing elements 406 comprise a plasmonic material such as, but not limited to, gold, silver, copper, platinum, aluminum, etc., or a combination of these metals in the form of alloys, or other suitable material that is able to support surface plasmons for field enhancement for Raman scattering. In addition, the Raman-enhancing elements 406 may be multilayer structures, for example, 10 to 100 nm silver layer with 1 to 50 nm gold over-coating, or vice versa. By definition herein, a plasmonic material is a material that supports plasmons.

Turning now to FIG. 4B, there is shown a cross-sectional view along a line A-A, shown in FIG. 4A, of the array 400, in accordance with an example. As shown therein, each of the tips 408 of the nano-fingers 404 includes a respective Raman-enhancing elements 406 disposed thereon. The Raman-enhancing elements 406, which may comprise metallic nanoparticles, may be deposited onto the tips 408 of the nano-fingers 404 through one of, for instance, physical vapor deposition (PVD), chemical vapor deposition (CVD), sputtering, etc., of metallic material, or self-assembly of pre-synthesized nano-particles.

Although the nano-fingers 404 have been depicted in FIGS. 4A-4B as each extending vertically and at the same heights with respect to each other, it should be understood that some of the nano-fingers 404 may extend at various angles and heights with respect to each other. The differences in angles and/or heights between the nano-fingers 404 may occur, for instance, due to differences arising from manufacturing or growth variances existent in the fabrication of the nano-fingers 404 and the deposition of the Raman-enhancing elements 406 on the nano-fingers 404, etc.

As shown in FIG. 4B, the nano-fingers 404 are in a first position, in which the tips 408 are in a substantially spaced arrangement with respect to each other. The gaps 410 between the tips 408 may be of sufficiently large size to enable a liquid to be positioned in the gaps 410. In addition, the gaps 410 may be of sufficiently small size to enable the tips 408 of at least some of the nano-fingers 404 to be drawn toward each other as the liquid provided in the gaps 410 evaporates, through, for instance, capillary forces applied on the tips 408 as the liquid evaporates.

Turning now to FIG. 4C, there is shown a cross-sectional view along a line A-A, shown in FIG. 4A, of the array 400, following evaporation of the liquid, according to an example. The view depicted in FIG. 4C is identical to the view depicted in FIG. 4B, except that the nano-fingers 404 are depicted in a second position, in which the tips 408 of some of the nano-fingers 404 have been drawn toward with each other. According to an example, the tips 408 of some of the nano-fingers 404 may be in and may remain in relatively close proximity to with each other for a period of time due to the capillary forces applied on adjacent ones of the nano-fingers 404 during and following evaporation of the liquid (not shown) in the gaps 410 between the tips 408.

In any event, and in one regard, the tips 408 of the nano-fingers 404 are caused to be drawn toward each other as shown in FIG. 4C to enhance Raman signal emission by analyte molecules 412 in the near fields of the Raman-enhancing elements 132 because the relatively small gaps between the Raman-enhancing elements 132 on the adjacent tips 136 create “hot spots” having relatively large electric field strengths.

According to an example, the nano-fingers 404 are positioned into the collapsed state depicted in FIG. 4C prior to being positioned substantially in the focal point of the parabolic reflector 102. In addition, the array 400 is inverted, such that the Raman-enhancing elements 406 are directed toward the parabolic reflector 102. It should also be noted that the Raman-enhancing elements 406 may comprise other nanostructures and nanoparticles that are coated with a plasmonic material such as metal. In these examples, the Raman-enhancing elements 406 may comprise, for instance, gold and silver colloidal nanoparticles, black silicon coated with Au or Ag, etc.

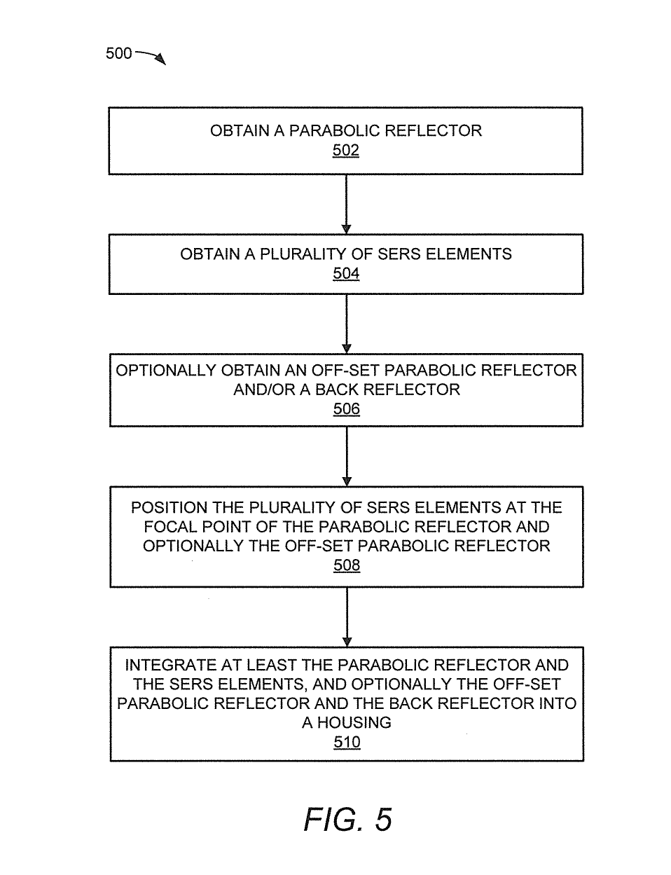

Turning now to FIG. 5, there is shown a flow diagram of a method 500 for fabricating an apparatus for performing spectroscopy, according to an example. It should be understood that the method 500 depicted in FIG. 5 may include additional processes and that some of the processes described herein may be removed and/or modified without departing from a scope of the method 500. In addition, although particular reference is made herein to the apparatus 100 as being fabricated through implementation of the method 500, it should be understood that the method 500 may be implemented to fabricate a differently configured apparatus without departing from a scope of the method 500.

At block 502, a parabolic reflector 102 is obtained. The parabolic reflector 102 may be obtained through fabrication of the parabolic reflector 102 in any of the manners discussed above. Alternatively, the parabolic reflector 102 may comprise a pre-fabricated component and may thus be obtained from a manufacturer or supplier of the parabolic reflector 102.

At block 502, a plurality of SERS elements 104 are obtained. The SERS elements 104 may be obtained through fabrication of the SERS elements 104 fabricated in any of the manners discussed above. Alternatively, the SERS elements 104 may be pre-fabricated on a substrate and may thus be obtained from a manufacturer or supplier of the SERS elements 104. According to a particular example, the SERS elements 104 comprise the Raman-enhancing elements 406 provided on the tips 408 of nano-fingers 406, as shown in FIG. 4C.

At block 506, an off-set parabolic reflector 202 and/or a back reflector 204 are optionally obtained. The obtaining of the off-set parabolic reflector 202 and/or the back reflector 204 is considered optional because the apparatus 100 may be fabricated without either of these reflectors. In the instance that either or both of these reflectors are obtained, the off-set parabolic reflector 202 and/or a back reflector 204 may be obtained through fabrication of these reflectors in any of the manners discussed above or obtained from a manufacturer or supplier of these reflectors. According to a particular example, the off-set parabolic reflector 202 is integrally formed with the parabolic reflector 102 as discussed above with respect to FIG. 2.

At block 508, the plurality of SERS elements 104 is positioned substantially at the focal point of the parabolic reflector 102. According to an example, the positioning of the SERS elements 104 with respect to the parabolic reflector 102 may occur during the obtaining of the parabolic reflector 102 and the SERS elements 104. Thus, for instance, the SERS elements 104 may be fabricated at block 504 to be positioned substantially at the focal point of the parabolic reflector 102. In any regard, the SERS elements 104 may be supported substantially at the focal point of the parabolic reflector 102 through use of any suitable mechanism, such as the supporting membrane 304 depicted in FIGS. 3A and 3B.

In addition, at block 508, if the off-set parabolic reflector 202 is to be included in the apparatus 100, the SERS elements 104 may also be positioned substantially at the focal point of the off-set parabolic reflector 202. Moreover, if the back reflector 204 is to be included in the apparatus 100, the back reflector 204 may be positioned to direct light emitted from the SERS elements 104 toward the parabolic reflector 102. Furthermore, the SERS elements 104 may be positioned substantially at the focal point of the back reflector 204.

At block 510, the parabolic reflector 102 and the SERS elements 104 are integrated into a housing 302. The parabolic reflector 102 and the SERS elements 104 may be integrated into the housing 302 in any of the manners discussed above with respect to FIGS. 3A and 3B. In addition, the off-set parabolic reflector 202 and/or the back reflector 204 may be integrated into the housing 302 as also discussed above.

According to an example, the parabolic reflector 102 and the SERS elements 104 are formed and then positioned into the housing 302. In another example, the parabolic reflector 102 and the SERS elements 104 are formed directly in the housing 302. The off-axis parabolic reflector 202 and the back reflector 204 may also be fabricated in either of these manners.

Although described specifically throughout the entirety of the instant disclosure, representative examples of the present disclosure have utility over a wide range of applications, and the above discussion is not intended and should not be construed to be limiting, but is offered as an illustrative discussion of aspects of the disclosure.

What has been described and illustrated herein is an example along with some of its variations. The terms, descriptions and figures used herein are set forth by way of illustration only and are not meant as limitations. Many variations are possible within the spirit and scope of the subject matter, which is intended to be defined by the following claims—and their equivalents—in which all terms are meant in their broadest reasonable sense unless otherwise indicated.

What is claimed is:

1. An apparatus for performing spectroscopy comprising:
a parabolic reflector; and
a plurality of surface-enhanced Raman spectroscopy (SERS) elements spaced from the parabolic reflector and positioned substantially at a focal point of the parabolic reflector, wherein the parabolic reflector is to reflect Raman scattered light emitted from molecules in a near field generated by the plurality of SERS elements to substantially increase the flux of the Raman scattered light emitted out of the apparatus.

2. The apparatus according to claim 1, further comprising:
an off-axis parabolic reflector positioned to direct pump light onto the SERS elements.

3. The apparatus according to claim 2, wherein the off-axis parabolic reflector is attached to the parabolic reflector.

4. The apparatus according to claim 2, wherein the parabolic reflector is tilted with respect to the off-axis parabolic reflector to cause excitation light emitted onto the off-axis parabolic reflector to be at an angle that differs from an angle at which the Raman scattered light is reflected from the parabolic reflector.

5. The apparatus according to claim 1, further comprising:
a back reflector, wherein the plurality of SERS elements is positioned between the parabolic reflector and the back reflector, and wherein Raman scattered light emitted from molecules in the near field generated by the plurality of SERS elements is to be reflected from the back reflector onto the parabolic reflector.

6. The apparatus according to claim 5, wherein the plurality of SERS elements comprises a substrate, and wherein the back reflector is incorporated into the substrate.

7. The apparatus according to claim 5, wherein the plurality of SERS elements is positioned substantially at a focal point of the back reflector.

8. The apparatus according to claim 1, further comprising:
a housing through which light is to be transmitted; and
wherein the parabolic reflector and the plurality of SERS elements are at least one of positioned within and integrated with the housing.

9. The apparatus according to claim 8, wherein the housing is sized and formed of a material to enable the housing to be implanted into a specimen.

10. The apparatus according to claim 1, wherein the plurality of SERS elements comprises a plurality of a nano-fingers on which Raman-enhancing elements are attached to free ends of the nano-fingers.

11. The apparatus according to claim 10, wherein the Raman-enhancing elements on the free ends of at least two of the plurality of nano-fingers are in close proximity to each other.

12. A method for fabricating an apparatus for performing spectroscopy, the method comprising:
obtaining a parabolic reflector, wherein the parabolic reflector has a focal point;
obtaining a plurality of surface-enhanced Raman spectroscopy (SERS) elements; and
positioning the plurality of SERS elements at a location that is substantially at the focal point of the parabolic reflector.

13. The method according to claim 12, further comprising:
obtaining an off-axis parabolic reflector; and
wherein positioning the plurality of SERS elements further comprises positioning the plurality of SERS elements such that the plurality of SERS elements are also substantially at the focal point of the off-axis parabolic reflector.

14. The method according to claim 12, further comprising:
obtaining a back reflector; and
positioning the back reflector to position the plurality of SERS elements between the back reflector and the parabolic reflector and to reflect Raman scattered light emitted from molecules in the near field generated by the plurality of SERS elements onto the parabolic reflector.

15. The method according to claim 12, further comprising:
integrating the parabolic reflector and the plurality of SERS elements into a housing through which light is to be transmitted, wherein the housing is sized, configured, and formed of a material to be implanted into a specimen.
